(19) United States
(12) Patent Application Publication
(10) Pub. No.: US 2011/0009865 A1
(43) Pub. Date: Jan. 13, 2011

(54) BONE FIXATION USING AN INTRAMEDULLARY PIN

(76) Inventor: Robert M. Orfaly, Lake Oswego, OR (US)

Correspondence Address: KOLISCH HARTWELL, P.C., 200 PACIFIC BUILDING, 520 SW YAMHILL STREET, PORTLAND, OR, 97204, US

(21) Appl. No.: 12/835,613

(22) Filed: Jul. 13, 2010

Related U.S. Application Data
(60) Provisional application No. 61/225,137, filed on Jul. 13, 2009.

(51) Int. Cl. A61B 17/58

(52) U.S. Cl. 606/62

(57) ABSTRACT

System, including methods, apparatus, and kits, for fixation of a bone, such as the clavicle, using an intramedullary pin. The pin may be threaded in a leading region of the pin and may define one or more transverse apertures in the trailing region of the pin to receive one or more fasteners. In some embodiments, the system may include the pin and a jig for installation of the pin. The jig may include a sleeve that connects to the pin and is configured to be advanced toward the pin and against bone to compress the bone, to permit at least one fastener to be placed into a transverse aperture of the pin while the bone is compressed.

### CROSS-REFERENCE TO PRIORITY APPLICATION

This application claims the benefit under 35 U.S.C. §119(e) of U.S. Provisional Patent Application Ser. No. 61/225,137, filed Jul. 13, 2009, which is incorporated herein by reference in its entirety for all purposes.

### INTRODUCTION

The clavicle is an S-shaped bone, alternatively referred to as a collarbone, that represents the only direct skeletal connection between the arm and the chest wall. As such, the clavicle is critical to maintaining the appropriate position of the shoulder in relation to the chest and provides a mechanical strut across which muscles that motor the shoulder exert force. The clavicle is a subcutaneous bone, connecting the sternum and scapula, with a paucity of soft tissue attachments. The most important of these attachments is the confluence of the deltoid and trapezial muscular attachments, referred to as the deltotrapezial fascia.

Fractures of the midportion of the clavicle (the “clavicular shaft”) are common injuries in adults, commonly resulting from a lateral fall onto the shoulder. These fractures typically are displaced, with the lateral fragment pulled inferiorly and anteriorly by the weight of the arm. The degree of displacement is most affected by the degree to which the overlying deltotrapezial fascia is stripped off of the bone. There is also commonly an inferior butterfly fragment of comminution, though all degrees of comminution (i.e., reduction to small pieces or particles) are seen, from none to extensive.

Clavicle fractures are well-known to be very slow to unite due to several factors. These include the typical degree of fracture displacement and comminution, the small diameter of the bone leading to small fracture surfaces, the poor periosteal blood supply resulting from the limited muscular attachments, the significant forces crossing the bone, and the difficulty in clinically achieving splinting to minimize fracture motion. As a result, prolonged time to union and bony nonunion are common. The historical consensus had been that this fracture would generally heal eventually and that even nonunions lead to good function. This situation, euphemistically referred to as a “fibrous union,” was thought to lead to an outcome similar to bony union. However, greater scrutiny has led to a shifting consensus that these injuries can lead to prolonged disability as one awaits fracture healing and that nonunions commonly remain painful, particularly with overhead use. In one study, clavicle fractures treated conservatively have been found to manifest an overall nonunion rate of 6.2% at 24 weeks follow-up. Fractures of the diaphysis were more likely to lead to nonunion in cases with displacement leading to lack of cortical contact, presence of comminution, female patients, and advanced age. A randomized control trial comparing nonoperative treatment to acute plate fixation demonstrated that faster and more reliable healing of a clavicle fracture is achieved with plate fixation. The most common reason for late operation in the surgically treated group was hardware irritation requiring removal. As well, the concept of a symptomatic malunion has been widely accepted when greater than two centimeters of shortening leads to a medially and anteriorly displaced shoulder and altered mechanics in shoulder elevation.

As a result of these considerations, fixation of displaced clavicle fractures has become much more widely practiced in recent years. Surgical treatment currently consists of open reduction of the fracture and fixation either with an intramedullary pin or a plate.

Intramedullary pinning generally involves inserting a pin (e.g., the Rockwood pin) antegrade through the lateral fragment, reducing the fracture, and passing the threaded medial end of the pin into the medial fracture fragment. The fracture may be stabilized by compressing a nut assembly over a lateral region of the pin against the outer cortex of the distal clavicle. Disadvantages of this mode of fixation include the common prominence of the lateral nut assembly, which sometimes is sufficient to cause skin breakdown. The Rockwood pin is only effective for treatment of fractures with good cortical apposition of the major fragments of the clavicle. Due to the requirement of compression in order to achieve stability, lack of or loss of cortical contact leads to displacement of the implant and loss of fixation. The Rockwood pin also requires open exposure of the fracture and some degree of soft tissue stripping for hardware placement in all cases.

Plate fixation of the clavicle can be utilized in a compressive mode when cortical contact allows. It can also be utilized in neutralization mode to position fragments appropriately without compression when excessive comminution is present. However, plate application typically requires broad exposure of the clavicle, which requires soft tissue stripping. The plate rests in a subcutaneous location and very commonly requires removal due to irritation. As well, the mechanical strength of the plate can be suboptimal in osteopenic bone and where the fracture pattern does not allow sufficient numbers of screws on one or both sides of the fracture.

Improved approaches to clavicle fixation are needed. An ideal clavicular fixation device may have a number of features. For example, the device may have sufficient mechanical stability for fractures in a wide range of positions across the diaphysis and in the presence of osteopenia. In addition, the device may be capable of compression when achievable but also of neutralization when comminution is present. Furthermore, the device may cause a minimum of prominence postoperatively, while retaining the ability to be easily removed when necessary. Additionally, minimally invasive insertion may be facilitated including insertion without exposure of the fracture when a closed or percutaneous reduction can be achieved.

### SUMMARY

The present disclosure provides a system, including methods, apparatus, and kits, for fixation of a bone, such as the clavicle, using an intramedullary pin. The pin may be threaded in a leading region of the pin and may define one or more transverse apertures in the trailing region of the pin to receive one or more fasteners. In some embodiments, the system may include the pin and a jig for installation of the pin. The jig may include a sleeve that connects to the pin and is configured to be advanced toward the pin and against bone to compress the bone, to permit at least one fastener to be placed into a transverse aperture of the pin while the bone is compressed.

### BRIEF DESCRIPTION OF THE DRAWINGS

FIG. 1 is a superior view of a fractured left clavicle fixed with an intramedullary pin that is attached to the clavicle with a threaded leading region of the pin and with threaded fasteners, in accordance with aspects of the present disclosure.

FIG. 2 is a side view of the pin of FIG. 1.

FIG. 3 is a longitudinal sectional view of the pin of FIG. 2, taken generally along line 3-3 of FIG. 2.

FIG. 4 is a sectional view of the clavicle, pin, and fasteners of FIG. 1, taken generally along line 4-4 of FIG. 1.

FIG. 5 is an anterior view of the fractured clavicle of FIG. 1 being reduced by an exemplary clamp assembly in preparation for insertion of the intramedullary pin of FIG. 1, in accordance with aspects of the present disclosure.

FIG. 6 is a side view of selected components of an exemplary fixation system including the pin of FIG. 1, an exemplary jig that attaches to the pin to facilitate installation of the pin in bone, and a drill bit and guide cannula extending through the jig and/or the pin, in accordance with aspects of the present disclosure.

FIG. 7 is a fragmentary view of a guide arm of the jig of FIG. 6, taken generally along line 7-7 of FIG. 6.

FIG. 8 is an exploded side view of the pin and jig of FIG. 6, with a guide arm of the jig removed, in accordance with aspects of the present disclosure.

FIG. 9 is a longitudinal sectional view of the jig of FIG. 8, taken generally along line 9-9 of FIG. 8.

FIG. 10 is an anterior view of a fractured clavicle being prepared to receive the pin of FIG. 1 by formation of a bore in the clavicle, during performance of an exemplary method of bone fixation, in accordance with aspects of present disclosure.

FIG. 11 is an anterior view of the clavicle of FIG. 10 as the pin of FIG. 1 is being driven into the bore of FIG. 10 by rotation of the jig of FIG. 7 attached to the pin, during performance of an exemplary method of bone fixation, in accordance with aspects of present disclosure.

FIG. 12 is an anterior view of the clavicle of FIG. 10 taken as in FIG. 11 but after the pin of FIG. 1 has been advanced farther into the bore in the clavicle and with the pin functioning as a lag screw that achieves some compression of the clavicle during rotation of the pin with the jig of FIG. 7, during performance of an exemplary method of bone fixation, in accordance with aspects of present disclosure.

FIG. 13 is an anterior view of the clavicle of FIG. 10 as a sleeve of the jig of FIG. 7 is turned to advance the sleeve toward the pin and against the clavicle such that the clavicle is compressed more than in FIG. 12, during performance of an exemplary method of bone fixation, in accordance with aspects of present disclosure.

FIG. 14 is an anterior view of the clavicle of FIG. 10 as a guide arm of the jig of FIG. 6 is being connected to the body of the jig, during performance of an exemplary method of bone fixation, in accordance with aspects of the present disclosure.

FIG. 15 is an anterior view of the clavicle of FIG. 10 as a transverse hole is being formed in the clavicle, during performance of an exemplary method of bone fixation, in accordance with aspects of the present disclosure.

FIG. 16 is an anterior view of the clavicle of FIG. 10 as a threaded fastener is being placed into the transverse hole formed in FIG. 15, during performance of an exemplary method of bone fixation, in accordance with aspects of present disclosure.

FIG. 17 is a superior view of a multiply fractured clavicle being fixed with the pin of FIG. 1, with the pin connected to an extension member that extends away from the pin to engage the acromion of the scapula adjacent the clavicle, in accordance with aspects of the present disclosure.

### DETAILED DESCRIPTION

The present disclosure provides a system, including methods, apparatus, and kits, for fixation of a bone, such as the clavicle, using an intramedullary pin. The pin may be threaded in a leading region of the pin and may define one or more transverse apertures in the trailing region of the pin to receive one or more fasteners. In some embodiments, the system may include the pin and a jig for installation of the pin. The jig may include a sleeve that connects to the pin and is configured to be advanced toward the pin and against bone to compress the bone, to permit at least one fastener to be placed into a transverse aperture of the pin while the bone is compressed.

The pin may be an intramedullary pin structured to be placed along a medullary canal of the clavicle (or other bone (e.g., the ulna or fibula) and may include a head and an elongate body or shaft extending from the head toward a leading end of the pin. The head may be wider than the shaft and may include one or more transverse apertures for receiving threaded fasteners that secure the head to the clavicle (e.g., at an anatomical position that is lateral to a fracture in the clavicle). The transverse apertures may define axes that are parallel, orthogonal, or oblique to one another, and the transverse apertures each may (or may not) be locking apertures including an internal thread to provide threaded engagement with respective threaded fasteners, such as bone screws. The head also may define a longitudinal aperture extending coaxially with the head and/or shaft from a trailing end of the pin and providing an internal thread for attachment of a jig or insertion tool (e.g., a driver), an extension structure to increase the length of the pin, a compression assembly, a guide arm, or any combination thereof, among others. The body or shaft of the pin may be nominally cylindrical and may include an external thread disposed along at least (or only) a portion of the shaft length. For example, the external thread may be restricted to a leading section of the shaft, such that a nonthreaded trailing section of the shaft spans a discontinuity (such as a fracture) in the clavicle (or other bone) and the external thread secures the pin to the clavicle on an opposing side of the fracture from the pin's head (e.g., anatomically medial to the fracture). The external thread may be disposed adjacent a blunt tip that forms the leading end of the pin's body. The tip and/or pin may (or may not) define a cannulation for receiving a guide wire.

Further aspects of the present disclosure are included in the following sections: (I) exemplary pins for bone fixation, (II) exemplary instruments for pin installation, (III) exemplary methods of bone fixation and/or pin installation, (IV) composition of system components, (V) kits, and (VI) examples.

### I. EXEMPLARY PINS FOR BONE FIXATION

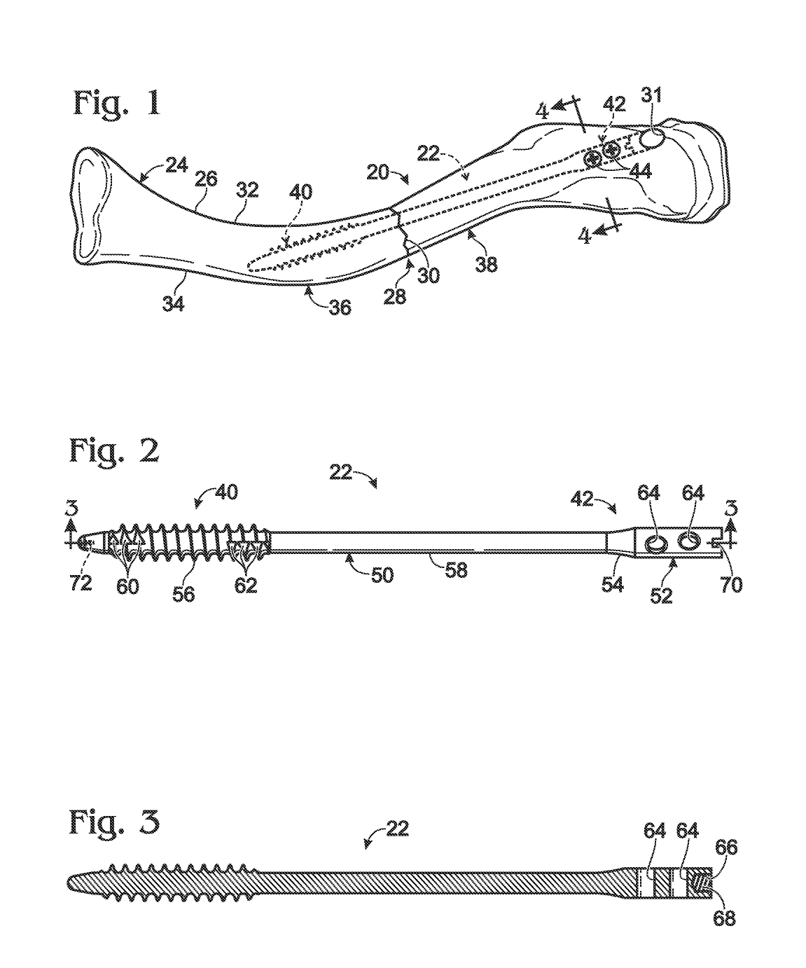

FIG. 1 shows selected components of an exemplary system 20 for bone fixation. The system may include an intramedullary pin 22, which may be installed longitudinally in a bone 24, such that the pin extends along the bone (e.g., along the medullary canal of the bone). Pin 22 may be disposed in any suitable bone 24, such as a clavicle 26 (or ulna or fibula, among others) that includes a discontinuity 28, for example, a fracture 30 or a cut. The pin may be described as an intramedullary nail or rod. The may (or may not) be disposed completely within the bone.

The pin in the present illustration is disposed within a fractured left clavicle. Pin 22 may be inserted into the clavicle to span fracture 30, by placement into and advancement through an entry hole 31 formed laterally in the clavicle. In this view taken from above the sternum, a medial end of the clavicle (which articulates with the sternum) is disposed on the left side of FIG. 1; a lateral end of the clavicle (which articulates with the acromion of the scapula) is disposed on the right side of FIG. 1. Also, respective posterior and anterior surface regions 32, 34 of the clavicle form upper and lower boundaries of the clavicle in this superior view.

Pin 22 may be secured to the clavicle. The pin may be attached to medial and lateral bone fragments 36, 38 of clavicle 26 that are disposed on opposing sides of fracture 30. For example, a leading region 40 of pin 22 may be disposed in threaded engagement with medial fragment 36. In addition, a trailing region 42 of the pin may be attached to lateral fragment 38 with fasteners 44, particularly threaded fasteners such as bone screws.

FIGS. 2 and 3 show respective side and sectional views of pin 22 in the absence of the clavicle and fasteners. The pin may include a shaft 50 connected to a head 52. The head may (or may not) be wider than the shaft. Accordingly, the pin may be described as having a wider head connected to a narrower shaft. In some embodiments, the pin may taper from head 52 to shaft 50, to form a tapered region 54 between the head and the shaft. In some examples, at least a portion (or all) of shaft 50 may be included in leading region 40 of the pin, and head 52 may form trailing region 42 of the pin.

Leading region 40 and trailing region 42 of the pin may include respective attachment features to permit the pin to be attached to bone and/or to a jig.

The leading region of the pin may be equipped with an external thread 56, which may be formed on shaft 50. External thread 56 may be formed on only a portion of the length of shaft 50, such as on only a leading section of the shaft. Accordingly, the shaft may include a nonthreaded trailing section 58 disposed between thread 56 and head 52. The nonthreaded trailing section of the shaft may be about as long as or longer than the threaded section of the shaft. A nonthreaded trailing section of the shaft may be advantageous for spanning the fracture, such that the external thread of the leading section does not engage fragments on opposing sides of the fracture, which may prevent compression. Alternatively, or in addition to a thread, the leading region of the pin may be equipped with a distinct type of attachment feature, such as fins, barbs, ribs, one or more transverse apertures to receive fasteners, or the like.

External thread 56 may be self-tapping. In particular, the thread may have one or more flutes 60, 62 for forming a thread in bone. In some embodiments, one or more trailing flutes 62 may be oriented rotationally opposite of one or more leading flutes 60, to facilitate removal of the pin.

The trailing region of the pin, such as head 52, may define at least one, at least two, or three or more transverse apertures 64 to receive fasteners that attach the trailing region of the pin to bone. The transverse apertures may define parallel axes or may define axes that are rotationally offset from one another about the longitudinal axis (and/or about a transverse axis) of the pin, such as offset by about 10 degrees from each other.

The trailing region (and/or head 52) may define mating structure to engage a jig. Head 52 may define a longitudinal aperture or socket 66 extending coaxially into the pin from its trailing end (see FIG. 3). Socket 66 (and/or any of the other apertures of the pin) may include (or lack) an internal thread 68. The head also may define a trailing notch 70 (see FIG. 2), which may orient a jig about the longitudinal axis of the pin, to facilitate alignment of a targeting guide of the jig with the transverse apertures.

Pin 22 may be structured to provide advantages over other intramedullary fixation devices for the clavicle. For example, a threaded leading end may provide stable fixation in the medial portion of the clavicle's medullary canal without the large diameter of shaft and rigidity required to allow for holes that receive transverse screws. It also may not be desirable to require transverse screws in the medial clavicle where major vascular, neurological, and pulmonary structures are within close proximity. Placement of transverse screws laterally in the clavicle, to attach head 52 to the clavicle, may be safer than placement medially in the clavicle, given the much greater distance of the lateral clavicle from neurovascular structures and the chest cavity.

The pin may include a recess or bore 72 formed in a leading tip of the pin. The recess or bore may extend only a short distance into the pin or may extend completely through the pin, between the leading and trailing ends thereof. In other words, the pin may or may not be cannulated, to permit the pin to be received on a guide wire.

FIG. 4 shows a sectional view of clavicle 26 and pin 22, with an exemplary fastener 44 extending through a transverse aperture 64 of the head, to attach the head to the clavicle. As shown here, the fastener may not (or may) be disposed in threaded engagement with the pin at the transverse aperture. In any event, the fastener may be a bone screw, such as a bicortical bone screw disposed in threaded engagement with opposing regions of the clavicle cortex (as shown somewhat schematically here), a unicortical bone screw that threads into only one cortical region, or a cancellous bone screw, among others. Fastener 44 may have a flat head 74, which may reduce the ability of the fastener's head to penetrate bone, while avoiding prominence of the head (and increased soft tissue irritation) in a subcutaneous location. The heads of threaded fasteners 44 may have a minimized thickness as these heads may be the only implanted parts of the fixation system that lie outside of bone when the pin is installed fully.

### II. EXEMPLARY INSTRUMENTS FOR PIN INSTALLATION

This Section describes exemplary instruments that be used to facilitate pin installation. These instruments may include an external fixator or clamp assembly and at least one jig, among others. The jig (or two or more distinct jigs), which may be termed an outrigger, may be utilized to drive the pin into bone, to compress bone, to guide formation of transverse holes and/or transverse fastener placement, or a combination thereof, among others.

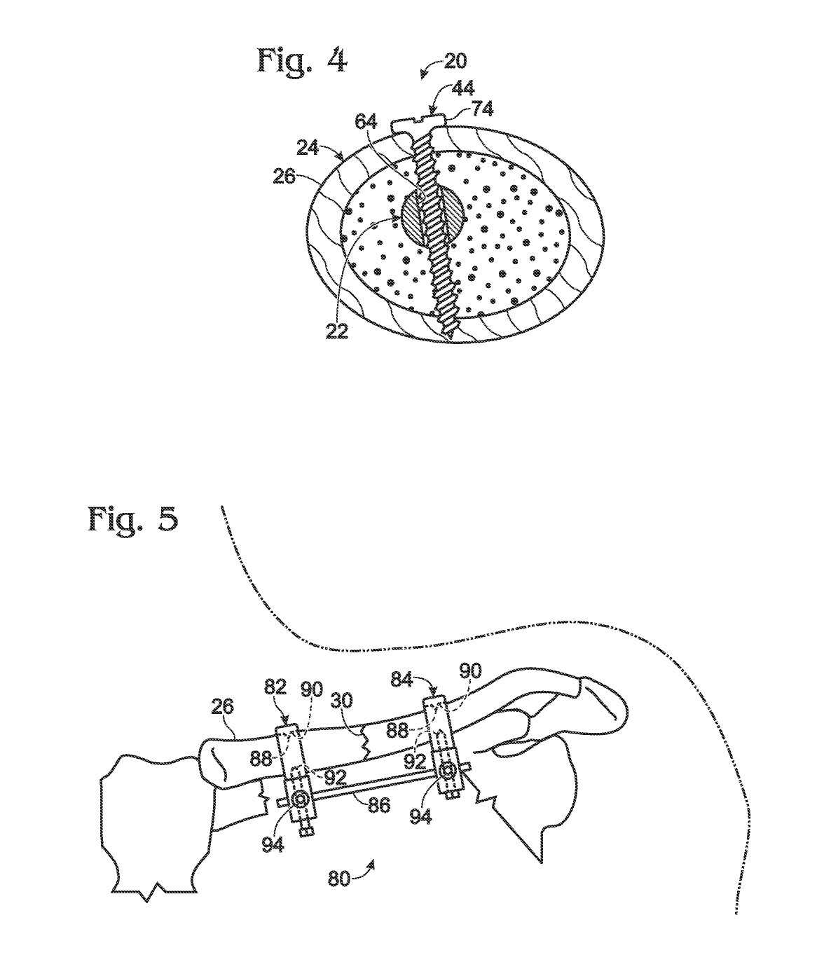

FIG. 5 shows an anterior view of clavicle 26 being reduced provisionally by an exemplary clamp assembly 80 in preparation for insertion of pin 22. Clamp assembly 80 may be an external fixator that functions percutaneously in fracture reduction. Due to percutaneous installation, clamp assembly 80 may avoid the need for open fracture exposure in most cases.

Clamp assembly 80 may include two or more C-shaped clips or clamp members 82, 84 connected by a spanning member 86. In use, a respective clamp member 82, 84 may be applied to each major fragment of the clavicle using at least two or three teeth 88-92 of the clamp member placed posterosuperiorly and anteroinferiorly over the clavicle. The teeth may be short and/or sharp to resist slippage on bone without damaging the bone excessively. Each clamp member may be resilient to facilitate application to the clavicle. Alternatively, or in addition, at least one of the teeth (e.g., tooth 92) may be adjustably positioned (e.g., by threaded engagement of the tooth with a frame of the clamp member) to provide adjustable engagement of the teeth with bone. These clamps then may be connected to spanning member 86 or another temporary external fixation device, which may assist in achieving and maintaining reduction while the pin is placed into the clavicle. The spanning member may be held in place with set screws 94 that permit that separation and relative angulation of the clamp members to be adjusted relative to one another.

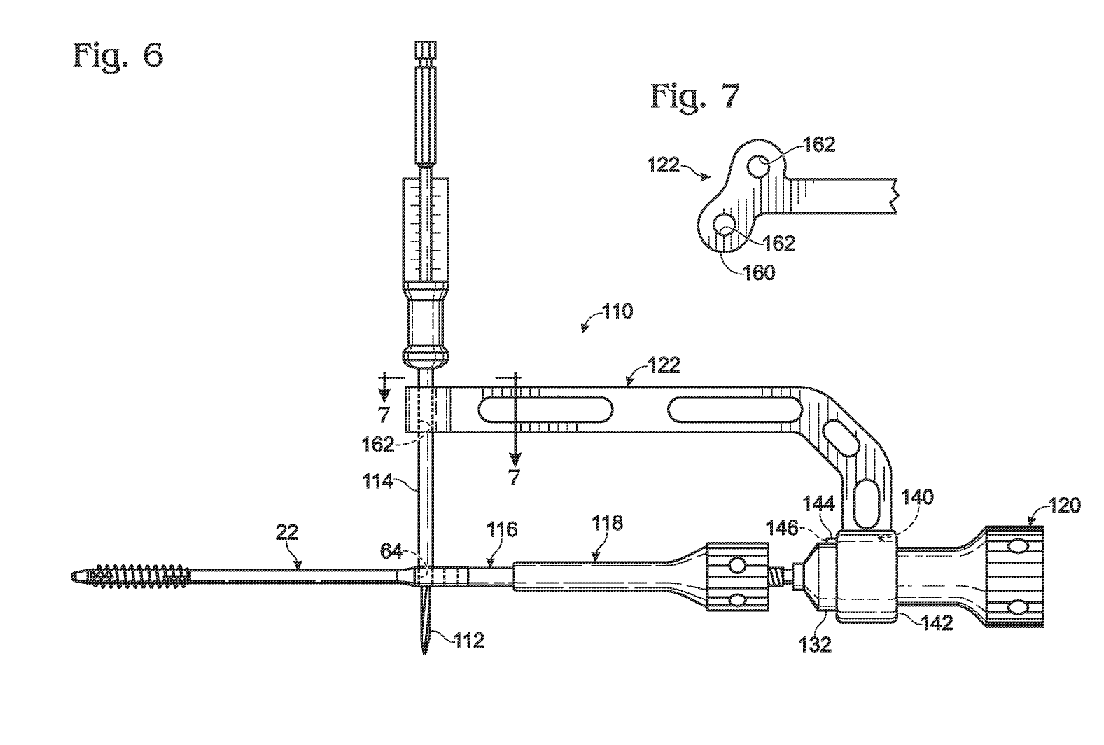

FIGS. 6 and 7 show a side view and a fragmentary view of selected components of an exemplary fixation system including (1) pin 22, (2) an exemplary jig 110 that attaches to the pin to facilitate installation of the pin in bone, and (3) a drill bit 112 and guide cannula 114 extending through the jig and/or a transverse aperture 64 the pin. Jig 110 may incorporate any combination of a body 116, a compression sleeve 118 in threaded engagement with the body, a drive knob 120, and/or a guide arm 122, among others.

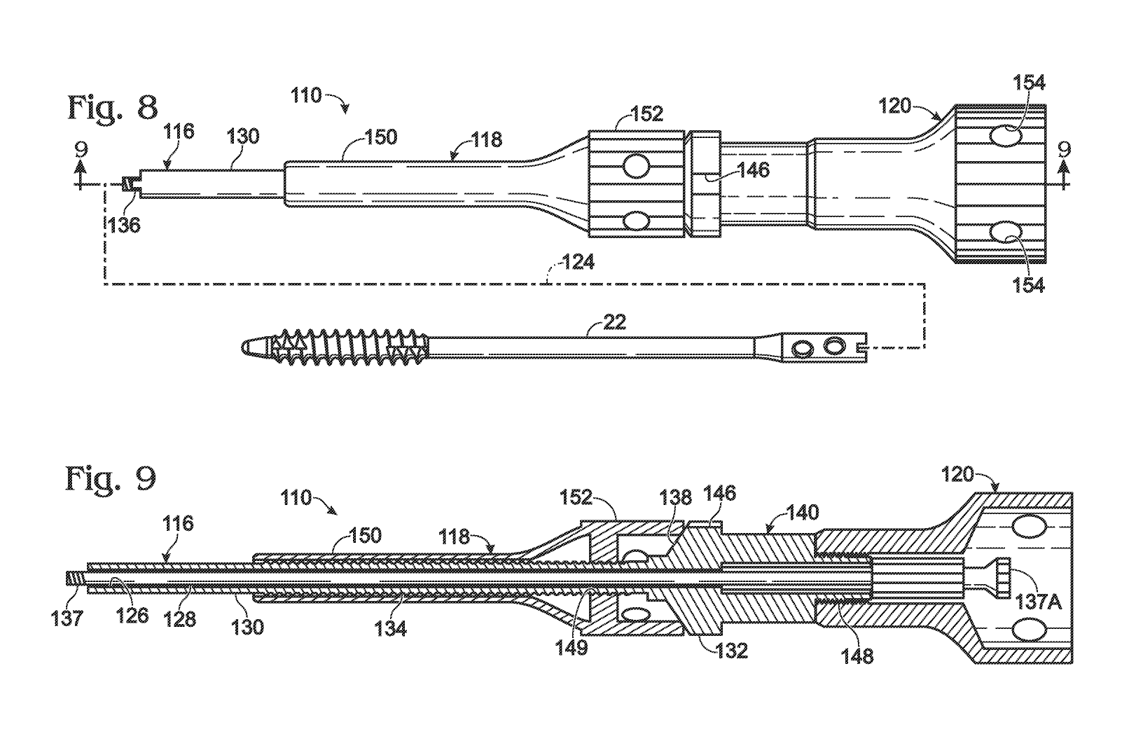

FIGS. 8 and 9 show respective side and sectional views of jig 110 with guide arm 122 removed. The jig attaches, indicated by a dot-dash line at 124 in FIG. 7, to pin 22, to enable installation of the pin in bone.

Body 116 of the jig may have any suitable structure. The body may be elongate and may be hollow or cannulated, to define a longitudinal channel 126 to accept a connecting rod 128 of the jig (see FIG. 9). Body 116 may include a stem 130 connected to a head 132.

Stem 130 may extend from head 132 to pin 22 when the jig is attached to the pin. The stem may include an external thread 134, which may be formed along any suitable portion of the length of the stem. Generally, the longitudinal extent of thread 134 determines how far compression sleeve 118 can travel along the stem. The stem also may include at least one tab 136 that mates with notch 70 of pin 22, to establish a predefined angular disposition of body 116 with respect to pin 22, and particularly with respect to transverse apertures 64 (see FIGS. 2 and 8). With tab 136 disposed in notch 70, connecting rod 128, which has a threaded tip 137, may be screwed into socket 66 of pin 22 to secure body 116 of the jig to pin 22 (see FIGS. 3, 8, and 9). An opposing end of rod 128 is equipped with a handle 137A that allows rod 128 to be turned manually, and pushed in to and pulled out of channel 126.

Head 132 may be fixed to stem 130, such that rotation of head 132 also rotates the stem. Head 132 may form a shoulder 138 that limits travel of sleeve 118 away from pin 22. Also, head 132 may form a mounting region 140 that mates with guide arm 122, to mount the guide arm on body 116. In particular, the guide arm may form a ring 142 that is received over a narrowed region of head 132 (see FIGS. 6, 8, and 9). The guide arm also may mate at only one predefined rotary position (or interchangeably at one of a pair of rotary positions related to one another by one-half turn). For example, the guide arm may provide a key 144 that fits into a slot 146 defined by mounting region 140 (see FIGS. 6 and 8). Furthermore, head 132 may provide a threaded post 148 at its trailing end to receive drive knob 120.

Compression sleeve 118 may be arranged coaxially with body 116 and pin 22. The sleeve may be driven along stem 130 by turning the sleeve, which may include an internal thread 149 that matches and mates with external thread 134 of body 116 (e.g., the thread formed on stem 130). The sleeve may taper toward the pin, to form a narrowed, elongate nose 150 projecting from a wider sleeve handle 152 (see FIG. 8). The nose may, for example, have a diameter that is less than about twice that of the pin's head, to facilitate advancement of the nose through an incision in soft tissue to contact bone. Sleeve handle 152 may be engaged manually (e.g., with a gloved hand) when turned and may include a knurled perimeter to enable grasping the handle. Compression sleeve 118, and particularly a leading end of nose 150, may be configured to be advanced toward pin 22 by any suitable distance, such as advanced to a position near the trailing end of the pin and/or overlapping the pin, among others.

The jig may include a drive handle, such as drive knob 120, which may be configured to be grasped manually (e.g., with a gloved hand), to rotate the jig's body and its attached pin, thereby driving the pin into bone. In other words, torque may be applied to the jig via the drive handle. Knob 120 may define one or more through-holes 154 to receive a lever, which may be used to increase the amount of torque that can be applied by a surgeon (see FIG. 8). In some examples, axial force (e.g., a continuous axial force or pulses of axial force, such as slaps or blows) may be applied to the drive handle, such with a hand or a mallet, among others. The drive knob also or alternatively may function as an end cap. In this role, the drive knob may restrict removal of guide arm 122 from mounting region 140 (e.g., by engagement of ring 142 of the guide arm; see FIG. 6) and/or may at least partially cover or enclose a trailing region of connecting rod 128 (see FIG. 9).

FIG. 7 shows a fragmentary view of guide arm 122 taken from above a distal end region 160 of the guide arm. The end region may define a plurality of openings 162 that each are coaxial with a different one of the transverse apertures of pin 22. Openings 162 may receive a drill bit 112 and a guide cannula 114 (see FIG. 6), and/or a driver to promote placement of fasteners into and/or through the transverse apertures of the pin.

### III. EXEMPLARY METHODS OF BONE FIXATION AND/OR PIN INSTALLATION

The present disclosure provides methods of bone fixation and/or pin installation into bone. The method steps described in the present disclosure may be performed in any suitable combination, in any suitable order, and each step may be repeated any suitable number of times. The remainder of this Section describes exemplary method steps that may be performed. The method steps of this Section may be combined with, modified by, or used with any of the procedures or apparatus described elsewhere in the present disclosure.

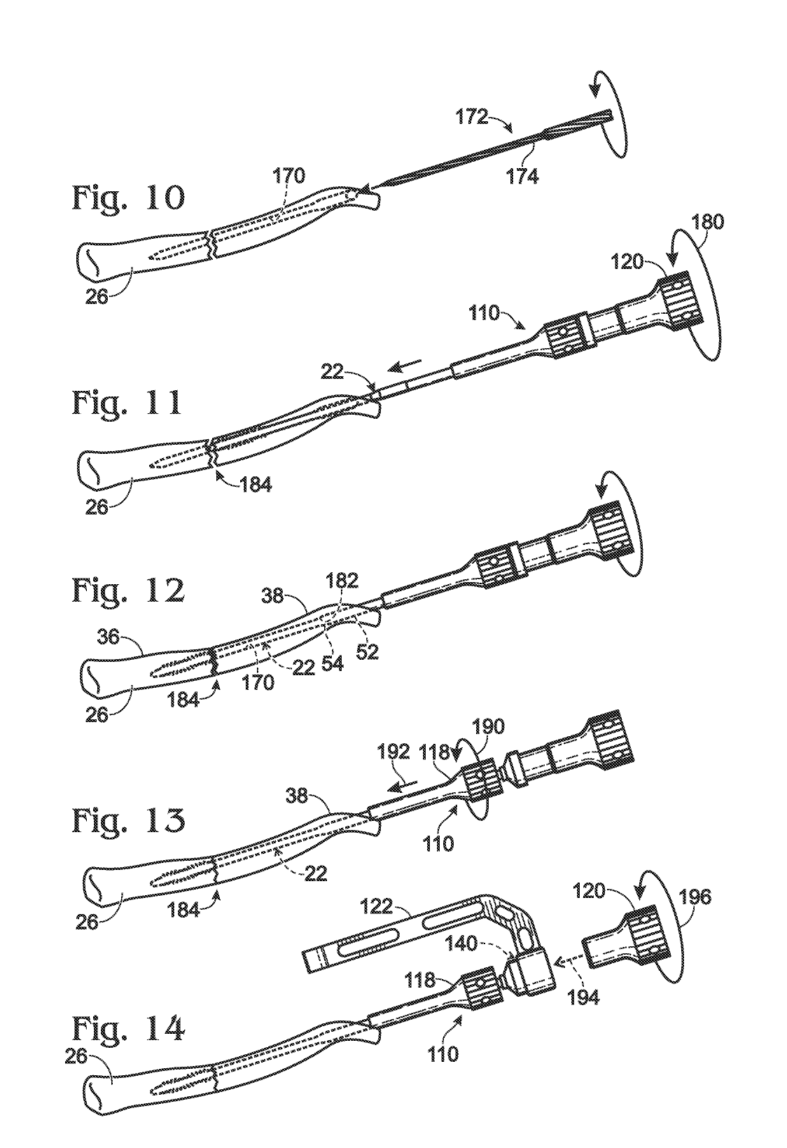

FIGS. 10-16 illustrate configurations that may be produced by performance of an exemplary method of bone fixation. These figures depict fixation of a fractured clavicle and are referenced in the remainder of this Section to exemplify, support, and clarify the more general description of the method presented herein.

A bone, such as a long bone, may be selected for fixation and/or to receive a pin. Exemplary long bones that may be suitable include a clavicle, a bone of the arms (such as a humerus, a radius, or an ulna), a bone of the legs (such as a femur, a tibia, or a fibula), a metacarpal, a metatarsal, a phalange, or the like. Bones that may be particularly suitable are a clavicle, a fibula, or an ulna.

The bone selected may have any suitable indication. Exemplary indications include at least one discontinuity present in the bone or to be introduced into the bone surgically, such as one or more fractures, cuts, nonunions, or a combination thereof, among others. The bone may include portions that are disposed on opposite sides of the discontinuity (or one of the discontinuities). Stated differently, the discontinuity may define portions of the bone, which may opposingly flank the discontinuity. Each portion may, for example, be a fragment, a collection of two or more fragments, a segment, and/or a region of the bone. The portions may be arranged generally end to end, that is, longitudinally along the bone.

A pin for fixation of the bone and/or installation into the bone may be selected and/or provided. The pin may include any combination of the features disclosed herein. For example, the pin may have an external thread, may be described as a rod or a nail, may define transverse apertures in leading and/or trailing regions of the pin, or any combination thereof, among others. The pin may be manufactured in a plurality of distinct diameters and/or a series of distinct lengths to permit a surgeon to select a suitable size of pin. The size of the pin may be selected according to the size of bone selected for fixation. For example, a longer and wider pin (e.g., an intramedullary nail) may be selected for a larger bone (such as a femur) and a shorter and narrower pin may be selected for a smaller bone (such as a clavicle, ulna, or fibula).

The bone may be prepared to receive the pin. For example, an incision may be made to access the bone, the bone may be provisionally fixed percutaneously, and/or a hole may be formed along the bone. The hole, which may be termed a bore, may correspond in length and/or diameter to the pin, or at least a portion of the bore may be formed as the pin is inserted into the bone.

FIG. 10 depicts an aspect of bone preparation and shows fractured clavicle 26 being prepared to receive a pin by formation of a bore 170 in the clavicle. The bore may be created by drilling, reaming, broaching, or a combination thereof, among others. For example, in the present illustration, a drill 172 with a stepped bit 174 is being used to form a narrower region and a wider region of the bore, to match the shape of the pin.

The clavicle may be prepared to receive the pin as follows, in some examples. With clamp assembly 80 provisionally fixing the clavicle (see FIG. 5), an incision (e.g., an incision of approximately one centimeter) may be made lateral and posterior to the acromioclavicular joint. Under fluoroscopic control, the posterior cortex of the clavicle then may be drilled, for example, approximately one centimeter medial to the acromioclavicular joint. This position corresponds to the apex of the posterior curvature of the lateral clavicle. A blunt-tipped hand reamer then may be used to open the medullary canal and cross the fracture site.

Alternatively, the pin may be capable of insertion associated with open reduction of the fracture. A two- to four-centimeter longitudinal incision along Langer's lines may be created over the tip of the medial fracture fragment. The fracture may be exposed and reamed medially and laterally. Laterally the cortex may be broached from within the canal.

The pin may be disposed or placed longitudinally in the bone. After placement into the bone, the leading region and the trailing region of the pin may be situated in respective portions of the bone disposed on opposing sides of a discontinuity (e.g., a fracture) in the bone, with the pin spanning the discontinuity. Placement of the pin into the bone may attach the pin to at least one portion of the bone, such as attachment of the leading region of the pin to a medial portion of the clavicle. Placement of the pin may include driving the pin into bone by application of axial force, turning force (torque), or both. For example, the pin may be pushed, tapped, or pounded into the bore formed in the bone until a thread of the pin encounters a narrower region of the bore. Then, the pin may be rotated, to provide threaded engagement and threaded advancement of the pin. Alternatively, the leading region of the pin (particularly if nonthreaded) may be attached to bone following placement into the bone, such as by installation of one or more fasteners into bone and into one or more transverse apertures defined by the leading region of the pin.

The pin may be equipped with a blunt tip on the leading end to aid in locating the entry site in the lateral clavicle. An insertion tool may assist in screwing the leading end of the pin into the medullary canal.

FIG. 11 exemplifies an aspect of pin introduction into bone. Pin 22 may be attached to jig 110 (e.g., lacking guide arm 122). The pin and the jig may be coaxial with one another. The jig may be engaged manually, such as via drive knob 120, and may permit manipulation of the pin via the jig, such as rotation of the jig and pin, indicated at 180. For example, jig 110 may function as a driver for manually turning pin 22 for threaded engagement with the clavicle, and/or for manually urging the pin longitudinally into (or out of) the medullary canal of the clavicle, as an aiming structure to determine the angular orientation of the pin as it is being inserted into an entry hole of the clavicle, and/or the like.

FIG. 12 depicts a compression of the clavicle (or other bone) that may be achieved by driving the pin into bone through rotation of the pin. When the pin is fully advanced into lateral fragment 38 of the clavicle, the trailing region of the pin (e.g., head 52 and/or tapered region 54) may engage a shoulder 182 formed in bore 170 of lateral fragment 38 that restricts further advancement of the trailing region of the pin with respect to lateral fragment 38. However, turning the pin may drive the leading region of the pin farther into medial fragment 36, which may compress the medial and lateral fragments together. As a result, a gap 184 between the fragments may be decreased or closed (compare FIGS. 11 and 12). Accordingly, pin 22 may function as a lag screw that urges bone portions together as the lag screw is turned.

The bone also or alternatively may be compressed with the jig, with the pin disposed in the bone, since using the pin as a lag screw may provide inadequate compression and/or may damage bone. Sleeve 118 of the jig may be turned, to advance the sleeve toward the pin and against a portion of the bone, such that the bone is compressed.

FIG. 13 exemplifies an aspect of bone compression with jig 110. Sleeve 118 of the jig of is being turned, indicated at 190, to advance the sleeve toward pin 22, in a direction indicated by an arrow at 192, and against lateral fragment 38 of clavicle 26. Pressing contact of sleeve 118 against fragment 38 (i.e., against the lateral clavicular cortex) may close gap 184 further, by urging the lateral fragment medially. Sleeve 118 may be larger in diameter than the head of the pin and of the bore in the clavicle through which the pin is inserted. The sleeve thus may serve to exert a compression force on the fracture site that the implanted pin then only has to maintain.

The trailing region of the pin may be secured to the bone while the sleeve compresses the bone. For example, one or more fasteners may be placed into one or more transverse apertures defined by the trailing region of the pin, to attach the trailing region to a portion of the bone while the bone is compressed by the sleeve. Before or during fastener placement, one or more holes may be formed in the bone that are coaxial with one or more transverse apertures defined by the trailing region of the pin. The fasteners may be placed into and/or through the transverse apertures via the one or more holes, or placement of the fasteners may form the holes in bone (if the fasteners are self-drilling).

FIG. 14 illustrates assembly of guide arm 122 with jig 110 in preparation for forming transverse holes in the bone that are coaxial with the transverse apertures of the pin. Drive knob 120 may be removed from the jig, guide arm 122 placed onto mounting region 140, and the drive knob re-connected to the jig, indicated at 194, 196, to hold the guide arm in place, while sleeve 118 compresses the clavicle.

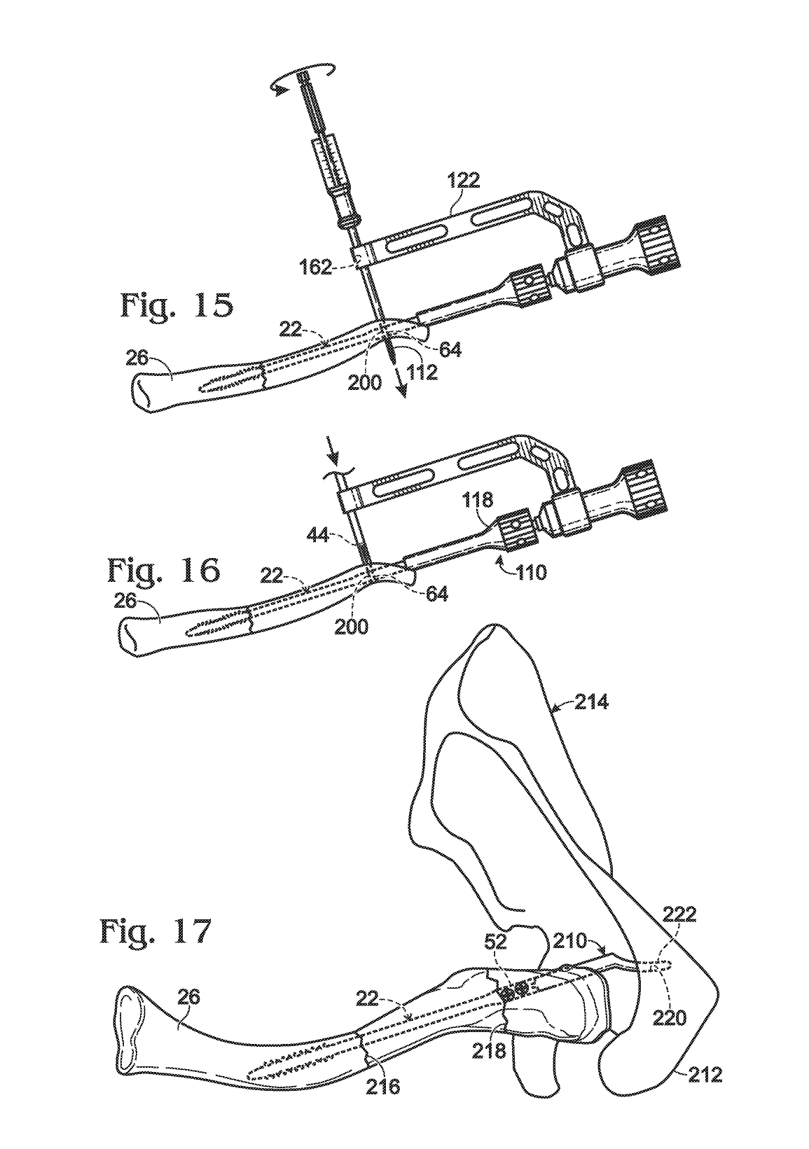

FIG. 15 illustrates formation of a transverse hole 200 in the clavicle that is coaxial with a transverse aperture 64 of the pin. Drill bit 112 extends coaxially from an opening 162 of guide arm and coaxially to transverse aperture 64. The transverse hole in bone may be formed according the rotary position of pin 22. For example, the transverse hole may be formed in a superior to inferior direction, as shown here, in an anterior to posterior direction, or the like. Placement of the transverse hole into the clavicle from a superior and slightly posterior starting point may assist in providing adequate soft tissue coverage for the fastener introduced into the transverse hole.

FIG. 16 shows threaded fastener 44 being placed into transverse hole 200 formed in FIG. 15. Each threaded fastener may be placed into a transverse aperture 64 of the pin from any suitable direction. After placement of one or more fasteners 44 to maintain the compression created by sleeve 118, jig 110 may be disconnected from pin 22 and removed. In some embodiments, threaded fasteners 44 can be placed without the use of compression sleeve 118, such as when the clavicle (or other bone) is comminuted and adequate cortical apposition of bone fragments cannot be achieved. In other words, the pin may be installed in a neutralization mode rather than a compression mode.

### IV. COMPOSITION OF SYSTEM COMPONENTS

Any components of the fixation system (i.e., pins, fasteners, jigs, extension attachments, etc.) may (or may not) be formed of any suitable biocompatible material(s) and/or bioresorbable (bioabsorbable) material(s). Illustrative biocompatible materials that may be suitable for a system component include (1) metals (for example, titanium or titanium alloy, cobalt-chrome alloy, stainless steel, etc.); (2) plastics or polymers (for example, ultra-high molecular weight polyethylene (UHMWPE), polymethylmethacrylate (PMMA), polytetrafluoroethylene (PTFE), polyetheretherketone (PEEK), and/or PMMA/polyhydroxyethylmethacrylate (PHEMA)); (3) ceramics (for example, alumina, beryllia, calcium phosphate, and/or zirconia, among others); (4) composites (for example, carbon-fiber composites formed with polymer and/or ceramic); (5) bioresorbable materials or polymers (for example, polymers of a-hydroxy carboxylic acids (e.g., polylactic acid (such as PLLA, PDLLA, and/or PDLA), polyglycolic acid, lactide/glycolide copolymers, etc.), polydioxanones, etc.); (6) bone material or bone-like material (e.g., bone chips, calcium phosphate crystals (e.g., hydroxyapatite, carbonated apatite, etc.); and/or the like. In some examples, one or more of these materials may form the body or core of a component and/or a coating thereon. Any two components of the fixation system may be formed of the same or different materials.

### V. KITS

The fixation system of the present disclosure, or any components thereof, may be provided in kits for bone fixation. The kits may include any combination of a pin or two or more pins (of the same or distinct lengths and/or diameters), one or more fasteners for securing the pin(s) to bone, a drill(s), a reamer, at least one jig, one or more clamp assemblies, a trailing attachment for the pin(s) to increase its length, and instructions for use. Some or all of the components of each kit may be provided in a sterile condition, such as packaged in a sterile container.

### VI. EXAMPLES

The following examples describe selected aspects and embodiments of the present disclosure, particularly exemplary systems for bone fixation using an intramedullary pin. These examples and the various features and aspects thereof are included for illustration and are not intended to define or limit the entire scope of the present disclosure.

### Example 1

### Exemplary Pin with Optional Extension Member

FIG. 17 shows a superior view of a multiply fractured clavicle 26 being fixed with pin 22, with the pin secured to an extension member 210. The extension member extends away from the pin to engage acromion 212 of a scapula 214 adjacent the clavicle. The extension member may be an optional attachment extending from the trailing end of the pin. Use of the extension member may be suitable when the pin is installed in a fractured clavicle that has multiple fractures 216, 218, with at least one of the fractures overlapping head 52 of the pin. Extension member 210, which may be termed a lateral or trailing flange or hook, may be bolted or otherwise attached to the head of the pin. In cases of distal or segmental clavicle fractures in which distal screw fixation may be insufficient, this attachment may be used. Extension member 210 may pass inferior to the mid-portion of the acromion and may or may not extend into the acromion. The extension member may be secured to the acromion. Alternatively, the extension member may not be secured to the acromion, but may engage the acromion to resist superior displacement of the pin with respect to the acromion. The acromion itself may not need to be exposed as long as a small channel 220 is created in the acromion for receiving a trailing finger 222 of extension member 210.

### Example 2

### Selected Embodiments

This example describes selected embodiments of the present disclosure, presented without limitation as a series of numbered sentences.

1. A device for bone fixation, comprising: a pin configured to be placed longitudinally in a bone and including a wider head connected to a narrower shaft, the shaft having a thread formed thereon in a leading region of the pin to permit threaded engagement of the leading region with bone, and the head defining one or more transverse apertures to receive fasteners that attach a trailing region of the pin to bone.

2. The device of paragraph 1, wherein the pin tapers between the head and the shaft.

3. The device of paragraph 1, wherein the shaft includes a threaded portion having the thread formed thereon and a nonthreaded portion lacking the thread, and wherein the nonthreaded portion is disposed between the threaded portion and the head and is longer than the threaded portion.

4. The device of paragraph 1, wherein the head defines an axial bore having an internal thread.

5. The device of paragraph 1, wherein the head defines two or more transverse apertures.

6. The device of paragraph 1, wherein the head is shorter than the shaft.

7. The device of paragraph 1, wherein the head is shorter than a nonthreaded portion of the shaft disposed between the thread and the head.

8. The device of paragraph 1, wherein the head is about a same length as a threaded portion of the shaft having the thread formed thereon.

9. A system for bone fixation, comprising: (A) a pin including a head connected to a shaft, the head defining one or more transverse apertures and the shaft having a thread formed thereon; and (B) a jig including a body and a sleeve in threaded engagement with each other, the jig being configured to be attached to the head of the pin to permit rotation of the body that drives the pin into threaded engagement with bone, the sleeve being advanceable along the body toward the pin by turning the sleeve, thereby permitting compression of the bone by the sleeve with the shaft threaded into bone and before placement of one or more fasteners into the transverse apertures.

10. A system for bone fixation, comprising: (A) a pin including a leading region connected to a trailing region, the leading region including at least one feature to permit attachment of the leading region to bone and the trailing region defining one or more transverse apertures configured to receive one or more fasteners that attach the trailing region to bone; and (B) a jig including a body and a sleeve in threaded engagement with the body, the body being configured to be attached to the trailing region of the pin with the sleeve coaxial to the pin, the sleeve being configured to be advanced along the body toward the pin by turning the sleeve, to permit compression of the bone by the sleeve with the leading region attached to the bone and before placement of one or more fasteners into the transverse apertures.

11. The system of paragraph 10, wherein the pin includes a wider head connected to a narrower shaft.

12. The system of paragraph 11, wherein the shaft has a thread formed thereon to permit threaded engagement of the leading region with bone.

13. The system of paragraph 11, wherein the pin tapers between the head and the shaft.

14. The system of paragraph 11, wherein the shaft includes a threaded portion having the thread formed thereon and a nonthreaded portion lacking the thread, and wherein the nonthreaded portion is disposed between the threaded portion and the head and is longer than the threaded portion.

15. The system of paragraph 11, wherein the head defines an axial bore having an internal thread.

16. The system of paragraph 11, wherein the head defines two or more transverse apertures.

17. The system of paragraph 11, wherein the head is shorter than the shaft.

18. The system of paragraph 11, wherein the head is shorter than a nonthreaded portion of the shaft disposed between the thread and the head.

19. The system of paragraph 11, wherein the head is about a same length as a threaded portion of the shaft having the thread formed thereon.

20. The system of paragraph 10, wherein the jig is configured to be disposed in threaded engagement with the trailing region of the pin.

21. The system of paragraph 10, wherein the jig includes a detachable guide member configured to guide formation of a hole in bone such that the hole is coaxial with a transverse aperture.

22. The system of paragraph 10, wherein the sleeve tapers toward the pin when the body is attached to the trailing region of the pin.

23. The system of paragraph 10, wherein the sleeve includes a handle portion configured to be engaged manually for turning the sleeve by hand.

24. A method of bone fixation, comprising: (A) providing a pin having a wider head connected to a narrower shaft, the shaft having a thread formed thereon; (B) disposing the pin longitudinally in a bone that includes a discontinuity, with the thread in threaded engagement with the bone and with the head and the thread situated in respective first and second portions of the bone that are separated from one another by a discontinuity; and (C) placing one or more fasteners into one or more transverse apertures defined by the head to attach the head to the second portion of the bone.

25. The method of paragraph 24, wherein the step of disposing includes a step of placing the pin in threaded engagement with the first portion of the bone and not in threaded engagement with the second portion of the bone.

26. The method of paragraph 24, wherein the step of disposing includes a step of placing the pin into a clavicle from a lateral region of the clavicle toward a medial region of the clavicle.

27. A method of bone fixation, comprising: (A) disposing a pin longitudinally in a bone and in threaded engagement with the bone; (B) turning a sleeve that is connected coaxially to the pin, to advance the sleeve toward the pin and against a surface of the bone such that the sleeve pushes against the bone, thereby achieving compression of the bone; (C) placing one or more fasteners into one or more transverse apertures defined by the pin; and (D) disconnecting the sleeve from the pin.

28. A method of bone fixation, comprising: (A) disposing a pin longitudinally in a bone having a discontinuity that separates the bone into first and second portions, with a leading region of the pin attached to the first portion of the bone; (B) turning a sleeve that is connected coaxially to the pin, to advance the sleeve toward the pin and such that the sleeves pushes against the second portion of the bone, thereby achieving compression of the bone; (C) placing one or more fasteners into one or more transverse apertures defined by a trailing region of the pin; and (D) disconnecting the sleeve from the pin.

29. The method of paragraph 28, wherein the step of disposing includes a step of attaching the leading region of the pin to the first portion of the bone by threaded engagement of the pin with the bone.

30. The method of paragraph 28, wherein the step of disposing includes a step of attaching the leading region to the first of the bone by placing one or more fasteners into apertures defined by the leading region of the pin.

31. The method of paragraph 28, wherein the step of disposing includes a step of disposing a pin in a clavicle.

32. The method of paragraph 28, wherein the step of disposing includes a step of driving the pin into the bone by rotating a jig secured to the trailing region of the pin, and wherein the jig includes the sleeve.

33. The method of paragraph 28, wherein the sleeve tapers toward the pin to form a nose connected to a handle, and wherein the step of turning is performed with the handle grasped manually.

34. The method of paragraph 28, further comprising (1) a step of connecting a guide member to a jig that includes the sleeve and (2) a step of forming a hole in the second portion of the bone along a path defined by the guide member and coaxial with a transverse aperture.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

1. A method of bone fixation, comprising:
providing a pin having a wider head connected to a narrower shaft, the shaft having a thread formed thereon;
disposing the pin longitudinally in a bone that includes a discontinuity, with the thread in threaded engagement with the bone and with the head and the thread situated in respective first and second portions of the bone; and
placing one or more fasteners into one or more transverse apertures defined by the head to attach the head to the second portion of the bone.

2. The method of claim 1, further comprising a step of turning a sleeve that is connected coaxially to the pin, to advance the sleeve toward the pin such that the sleeve pushes against the second portion of the bone to compress the bone, and wherein the step of placing is performed while the bone is compressed by the sleeve.

3. The method of claim 2, further comprising (1) a step of making an incision to access the bone and (2) a step of disconnecting the sleeve from the pin after the step of placing one or more fasteners and before the incision is closed.

4. The method of claim 2, wherein the sleeve is included in a jig that is connected coaxially to the pin by threaded engagement of the jig with the pin, and wherein the step of disposing includes a step of driving the pin into the bone by manually turning the jig.

5. The method of claim 2, further comprising a step of forming a hole in the bone coaxially with a transverse aperture of the head, wherein the step of forming a hole is guided by a guide member that is supported by a jig that includes the sleeve.

6. The method of claim 1, wherein the step of disposing includes a step of disposing the pin in a clavicle.

7. The method of claim 6, wherein the step of disposing includes a step of placing the pin into the clavicle from a lateral region of the clavicle toward a medial region of the clavicle.

8. The method of claim 1, wherein the step of placing one or more fasteners includes a step of introducing one or more threaded fasteners into the transverse apertures such that the fasteners are in threaded engagement with the bone but not in threaded engagement with the transverse apertures.

9. The method of claim 1, wherein the first and second portions of the bone are separated by the discontinuity in the bone.

10. A method of bone fixation, comprising:
disposing a pin longitudinally in a bone, with a leading region and a trailing region of the pin situated in respective first and second portions of the bone that are separated by a discontinuity in the bone;
attaching the leading region of the pin to the first portion of the bone;
turning a sleeve that is connected coaxially to the pin, to advance the sleeve toward the pin and against the second portion of the bone such that the sleeve pushes against the second portion of the bone to compress the bone; and
placing one or more fasteners into one or more transverse apertures defined by the trailing region of the pin, to attach the trailing region to the second portion of the bone.

11. The method of claim 10, wherein the step of attaching is performed by the step of disposing through threaded engagement of the leading region of the pin with the first portion of the bone.

12. The method of claim 10, wherein the step of attaching includes a step of placing one or more fasteners into one or more transverse apertures defined by the leading region of the pin.

13. The method of claim 10, wherein the step of disposing includes a step of disposing a pin in a clavicle.

14. The method of claim 10, wherein the step of disposing includes a step of driving the pin into the bone by rotating a jig secured to the trailing region of the pin, and wherein the jig includes the sleeve.

15. The method of claim 14, wherein the jig forms a handle, and wherein the step of rotating is performing via the handle and with the handle grasped manually.

16. The method of claim 10, wherein the sleeve tapers toward the pin, when connected to the pin, to form a stem connected to a handle, and wherein the step of turning is performed by turning the handle with the handle grasped manually.

17. A system for bone fixation, comprising:
a pin configured to be placed longitudinally in a bone and including a wider head connected to a narrower shaft, the shaft having a thread formed thereon in a leading region of the pin to permit threaded engagement of the leading region with bone, and the head defining one or more transverse apertures to receive fasteners that attach a trailing region of the pin to bone.

18. The system of claim 17, further comprising a jig including a body and a sleeve in threaded engagement with the body, the body being configured to be attached to the trailing region of the pin with the sleeve coaxial to the pin, the sleeve being configured to be advanced along the body toward the pin by turning the sleeve, to permit compression of the bone by the sleeve with the leading region of the pin attached to the bone and before placement of one or more fasteners into the transverse apertures.

19. The system of claim 18, wherein the head is shorter than the shaft.

20. The system of claim 18, wherein the head is shorter than a nonthreaded portion of the shaft disposed between the thread and the head.
